(19) United States
(12) Patent Application Publication
(10) Pub. No.: US 2026/0158246 A1
(43) Pub. Date: Jun. 11, 2026

(54) BALLOON FOR BALLOON CATHETER

(71) Applicant: KANEKA CORPORATION, Osaka-shi, Osaka (JP)

(72) Inventors: Masahiro KOJIMA, Settsu-shi (JP); Takahisa HAMABUCHI, Osaka-shi (JP)

(73) Assignee: KANEKA CORPORATION, Osaka-shi, Osaka (JP)

(21) Appl. No.: 18/707,192

(22) PCT Filed: Oct. 27, 2022
(86) PCT No.: PCT/JP2022/040248

§ 371 Date: May 3, 2024

(30) Foreign Application Priority Data
Nov. 8, 2021 (JP) 2021-182088

(51) Int. Cl. A61M 25/10

(52) U.S. Cl. CPC A61M 25/104; A61M 25/1002; A61M 2025/1086

(57) ABSTRACT

Provided is a balloon for a balloon catheter that can easily secure the balloon to the lesion and prevent the balloon from slipping from the lesion while efficiently incising the lesion. A balloon (2) having a straight tubular part (23), proximal tapered part (22), proximal sleeve part (21), distal tapered part (24), and distal sleeve part (25), and having a balloon body (20) having a belt-like area (40) extending in the longitudinal axis direction (x), the belt-like area (40) having a first region (41) consisting of a protrusion part having a height (H) from the outer surface of the balloon body (20) and a second region (42) having a lower height than the height (H), and the surface roughness of the second region (42) being larger than the surface roughness of the first region (41).

### TECHNICAL FIELD

The present invention relates to a balloon for a balloon catheter.

### BACKGROUND ART

Diseases such as angina pectoris and myocardial infarction are caused by the formation of stenotic areas hardened by calcification and other factors in the inner walls of blood vessels. One of the treatments for these diseases is angioplasty, in which a balloon catheter is used to dilate the stenotic area. Angioplasty is a minimally invasive therapy that does not require an open chest procedure like bypass surgery and is widely used.

In angioplasty, it is sometimes difficult to dilate a stenosis that has hardened due to calcification and other factors with a standard balloon catheter. In some cases, while the method of dilating a stenosis by implanting an indwelling expansion device called a stent into the stenosis is also used, an ISR (In-Stent-Restenosis) lesion may occur after this treatment, in which the neointima of the vessel grows excessively and the vessel becomes stenotic again. The neointima in ISR lesions is soft and the surface is slippery, so a standard balloon catheter may cause the balloon to shift out of the lesion site during balloon dilation to damage the vessel.

Balloon catheters that can dilate a stenosis even in such calcified or ISR lesions include balloon catheters with a protrusion, blade or scoring element on the balloon to bite into the stenosis. For example, Patent document 1 discloses a balloon catheter having a balloon with a modified section of varying tensile strength and a stopper with high frictional resistance on the outer surface of the balloon. Patent document 2 discloses a balloon catheter having a plurality of spaced apart wedge-shaped incisional devises, wherein the incisional devices have a predetermined shape.

### RELATED ART DOCUMENT

### Patent Document

Patent Document 1: JP 2018-7810 A
Patent Document 2: JP 2018-528055 T

### SUMMARY OF THE INVENTION

### Problems to be Solved by the Invention

A balloon catheter dilates a lesion such as stenosis by delivering the balloon to the lesion, securing the balloon at the lesion, incising the lesion with a protrusion or other means of incision, and inflating the balloon. If the balloon is not properly secured to the lesion, the balloon slips and shifts its position from the lesion, resulting in damage to blood vessels other than the lesion or failure to dilate the lesion. Therefore, in the above Patent documents 1 and 2, attempts have been made to prevent the balloon from slipping by providing a stopper or wedge-shaped incision device, but there was room for improvement in order to efficiently incise the lesion while easily securing the balloon to the lesion. Accordingly, the purpose of the present invention is to provide a balloon for a balloon catheter that can easily secure the balloon to the lesion and prevent the balloon from slipping from the lesion while efficiently incising the lesion.

### Means for Solving the Problems

One embodiment of a balloon for a balloon catheter of the present invention that can solve the above problem is as follows.
[1] A balloon for a balloon catheter: comprising a straight tubular part, a proximal tapered part located proximal to the straight tubular part, a proximal sleeve part located proximal to the proximal tapered part, a distal tapered part located distal to the straight tubular part, and a distal sleeve part located distal to the distal tapered part; and a balloon body having an outer surface and an inner surface, and a belt-like area extending on the outer surface of the balloon body in a longitudinal axis direction of the balloon body; wherein the belt-like area includes, in a cross-section in a radial direction of the balloon body, at least one first region consisting of a protrusion part having a height H from the outer surface of the balloon body, and at least one second region having a height from the outer surface of the balloon body lower than the height H; the first region is disposed in the straight tubular part; and a surface roughness of the second region is larger than a surface roughness of the first region.

The balloon body has the belt-like area extending on the outer surface in the longitudinal axis direction, the belt-like area includes the first region consisting of the protrusion part having a height H and the second region having a height lower than the height H, and the surface roughness of the second region is larger than the surface roughness of the first region, and thus, the second region allows the balloon to be easily secured to the stenosis. Furthermore, since the height H of the first region is higher than the height of the second region, the stenosis can be efficiently incised by the first region. This enables the balloon for a balloon catheter in accordance with embodiments of the present invention to prevent the balloon from shifting from the stenotic area and to provide easy and safe treatment.

The balloon for a balloon catheter in accordance with embodiments of the present invention is preferably the following [2] to [10].
[2] The balloon for a balloon catheter according to [1], wherein the belt-like area includes at least one transition region connecting the first region and the second region, and a surface roughness of the transition region is larger than the surface roughness of the first region.
[3] The balloon for a balloon catheter according to [1] or [2], wherein the second region is disposed in at least one of the proximal sleeve part and the distal sleeve part.
[4] The balloon for a balloon catheter according to [3], wherein the belt-like area further includes a third region having a height from the outer surface of the balloon body higher than the height H of the protrusion part of the first region in a cross-section in the radial direction of the balloon body, and the balloon satisfies at least one of the following (1) and (2):
(1) at least a part of the proximal tapered part has the third region when the second region is disposed in the proximal sleeve part; and
(2) at least a part of the distal tapered part has the third region when the second region is disposed in the distal sleeve part.
[5] The balloon for a balloon catheter according to any one of [1] to [4], wherein the balloon satisfies at least one of the following (3) and (4):
(3) the second region is disposed from the proximal sleeve part to the proximal tapered part; and
(4) the second region is disposed from the distal sleeve part to the distal tapered part.
[6] The balloon for a balloon catheter according to any one of [1] to [5], wherein the second region is disposed in at least one of the proximal tapered part and the distal tapered part.

[7] The balloon for a balloon catheter according to any one of [1] to [6], wherein the balloon satisfies at least one of the following (5) and (6):
(5) the second region is disposed from the proximal tapered part to a proximal end part of the straight tubular part; and
(6) the second region is disposed from the distal tapered part to a distal end part of the straight tubular part.
[8] The balloon for a balloon catheter according to [1] or [2], wherein the balloon satisfies at least one of the following (7) and (8):
(7) the second region is disposed from the proximal sleeve part to the proximal tapered part, and further to a proximal end part of the straight tubular part; and
(8) the second region is disposed from the distal sleeve part to the distal tapered part, and further to a distal end part of the straight tubular part.
[9] The balloon for a balloon catheter according to any one of [1] to [8], wherein one or more of the second regions are disposed in the straight tubular part.
[10] The balloon for a balloon catheter according to any one of [1] to [9], wherein the belt-like area includes at least one transition region connecting the first region and the second region, and one or more of the transition regions are disposed in the straight tubular part.
[11] The balloon for a balloon catheter according to any one of [1] to [10], wherein the belt-like area has an inner protrusion part that projects inwardly in a cross-section in the radial direction from the inner surface of the balloon body.

### Effects of the Invention

According to the above balloon for a balloon catheter, the balloon body has the belt-like area extending on the outer surface in the longitudinal axis direction, the belt-like area includes at least one first region consisting of a protrusion part having a height H and at least one second region having a height lower than the height H, and the surface roughness of the second region is larger than the surface roughness of the first region, and therefore, the second region allows the balloon to be secured to the stenosis site while the first region, which consists of a protrusion part with a higher height than the second region, allows the stenosis to be incised. As a result, according to the above balloon for a balloon catheter, it becomes possible to prevent the balloon from shifting from the stenosis site and to easily perform safe treatment.

### BRIEF DESCRIPTION OF THE DRAWINGS

FIG. 1 is a side view of a balloon catheter in accordance with one embodiment of the present invention.

FIG. 2 is a plan view of a distal part including the balloon of the balloon catheter shown in FIG. 1.

FIG. 3 is a cross-sectional view along the III-III line in FIG. 2.

FIG. 4 is a cross-sectional view along the IV-IV line in FIG. 2.

FIG. 5 is a cross-sectional view along the V-V line in FIG. 2.

FIG. 6 is a variation of FIG. 3.

FIG. 7 is a perspective view of a distal side of the balloon shown in FIG. 6.

FIG. 8 is a variation of FIG. 7.

FIG. 9 is another variation of FIG. 3.

FIG. 10 is still another variation of FIG. 3.

FIG. 11 is a cross-sectional view of a balloon in the radial direction in accordance with still another embodiment of the present invention.

FIG. 12 is a perspective view of a parison before inflating in accordance with one embodiment of the present invention.

### MODE FOR CARRYING OUT THE INVENTION

Hereinafter, the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of the present invention, the dimensions may differ from the actual dimensions in some cases.

A balloon for a balloon catheter in accordance with embodiments of the present invention has a straight tubular part, a proximal tapered part located proximal to the straight tubular part, a proximal sleeve part located proximal to the proximal tapered part, a distal tapered part located distal to the straight tubular part, and a distal sleeve part located distal to the distal tapered part; the balloon body has an outer surface and an inner surface, and has a belt-like area extending on the outer surface of the balloon body in the longitudinal axis direction of the balloon body; the belt-like area includes, in a cross-section in the radial direction of the balloon body, at least one first region consisting of a protrusion part having a height H from the outer surface of the balloon body, and at least one second region having a height from the outer surface of the balloon body lower than the height H; the first region is disposed in the straight tubular part; and a surface roughness of the second region is larger than a surface roughness of the first region.

Thus, the balloon for a balloon catheter in accordance with embodiments of the present invention has the configuration where the balloon body has the belt-like area extending in the longitudinal direction on the outer surface, the belt-like area includes the first region consisting of a protrusion part having a height H and the second region having a height lower than the height H, and a surface roughness of the second region is larger than a surface roughness of the first region, so that the balloon can be easily secured to the stenosis site by the second region having a larger surface roughness. Furthermore, the height H of the first region consisting of a protrusion part is higher than the height of the second region, enabling the stenosis to be efficiently incised by the first region. In addition, the first region has a smaller surface roughness than that of the second region, suppressing resistance when the protrusion part of the first region bites into the stenosis, and the stenosis can be easily incised by the protrusion part. As a result, according to the balloon for a balloon catheter of embodiments of the present invention, it becomes possible to prevent the balloon from shifting from the stenosis site to efficiently incise the stenosis, easily making the treatment safe.

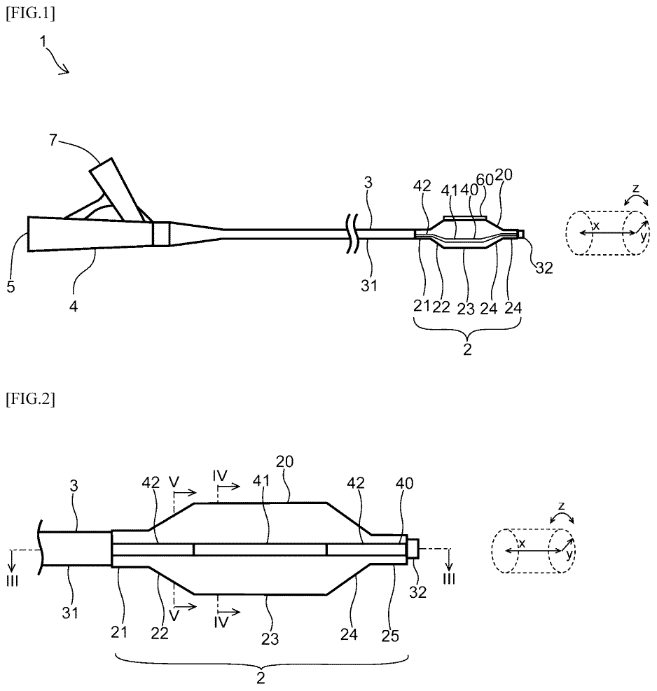

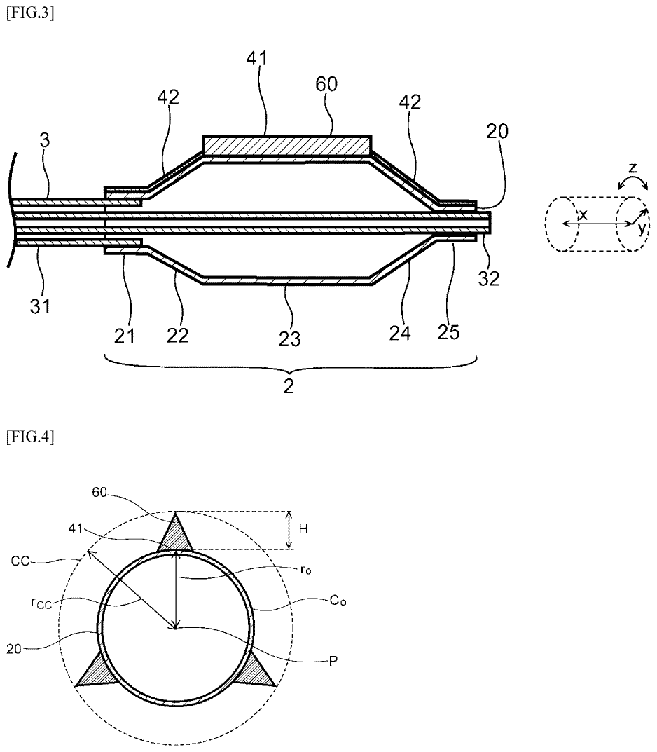

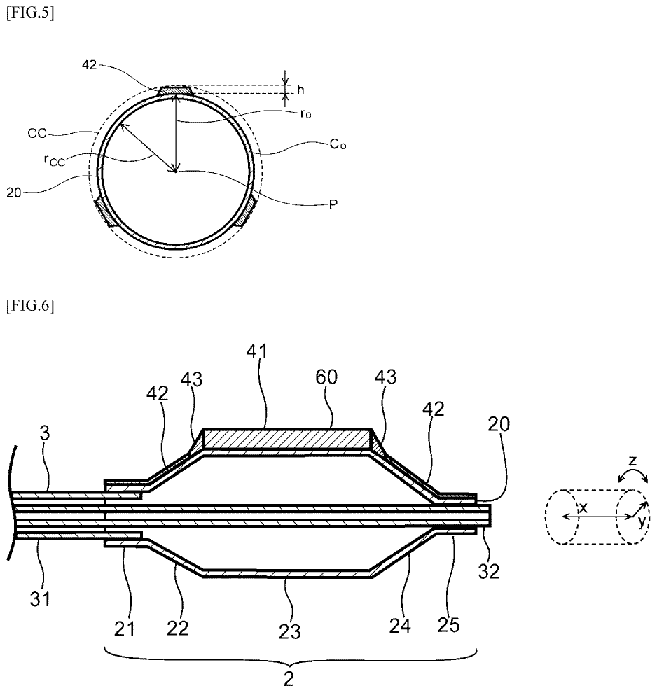

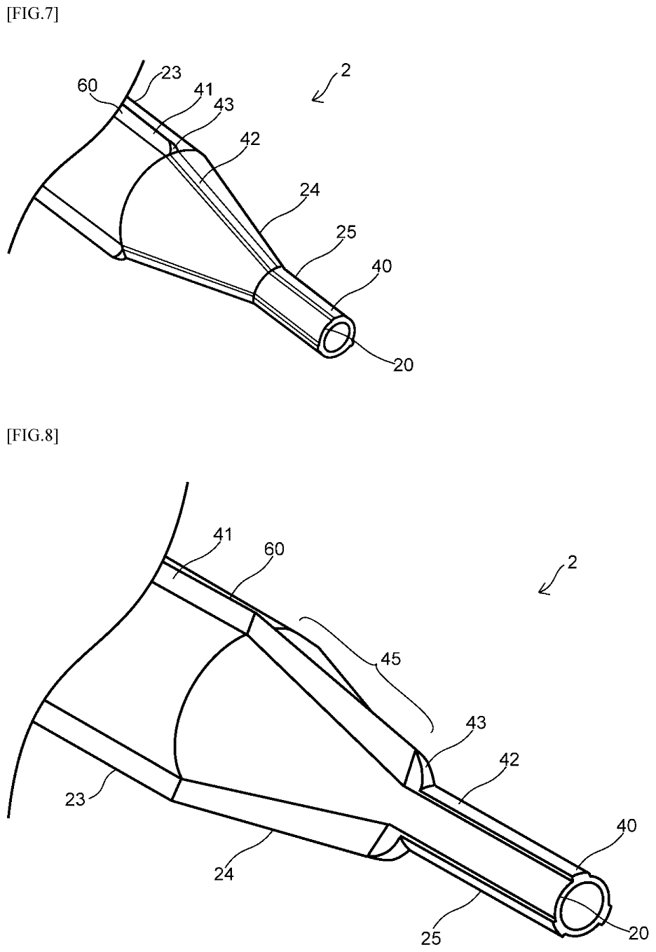

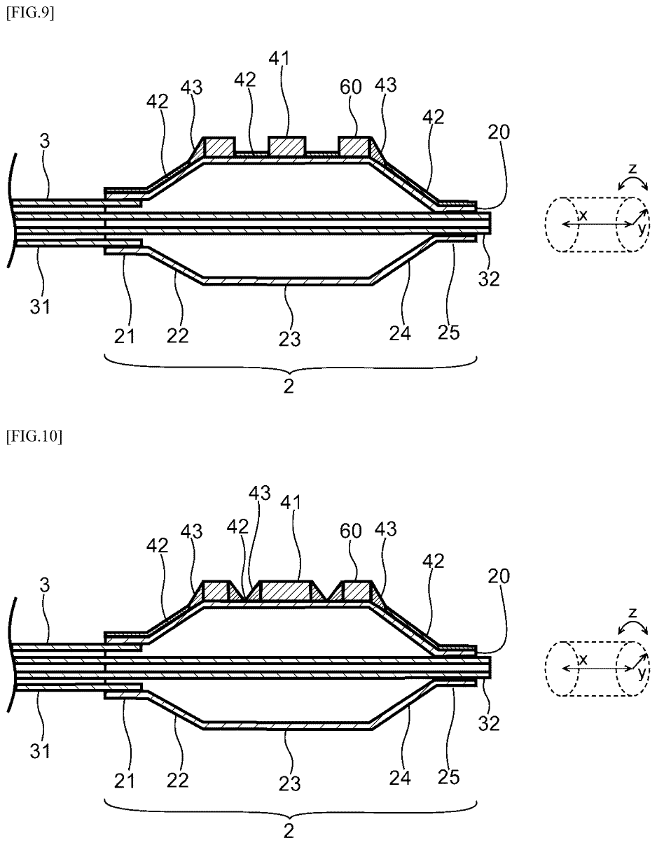

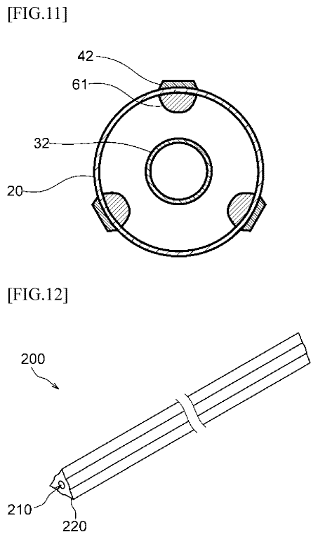

Referring to FIG. 1 to FIG. 11, the balloon for a balloon catheter in accordance with embodiments of the present invention will be described. FIG. 1 is a side view of a balloon catheter in accordance with one embodiment of the present invention. FIG. 2 is a plan view of a distal part including the balloon of the balloon catheter shown in FIG. 1. FIG. 3 to FIG. 5 are cross-sectional views along the III-III line, IV-IV line, and V-V line in FIG. 2, respectively. In FIG. 4 and FIG. 5, a shaft (inner tube) is omitted. FIG. 6 is a variation of FIG. 3, that is, a cross-sectional view of a distal part including a balloon for a balloon catheter in accordance with another embodiment of the present invention in the longitudinal axis direction, and FIG. 7 is a perspective view of a distal side of the balloon shown in FIG. 6. FIG. 8 is a variation of FIG. 7, that is a perspective view of a balloon in accordance with still another embodiment of the present invention. FIG. 9 and FIG. 10 are different variations of FIG. 3, respectively, that is, cross-sectional views in the longitudinal axis direction of a distal part including a balloon for a balloon catheter in accordance with different embodiments of the present invention, respectively. FIG. 11 is a cross-sectional view of a balloon in the radial direction in accordance with still another embodiment of the present invention. In this specification, a balloon for a balloon catheter may be simply referred to as “balloon”.

In the present invention, a proximal side refers to the direction towards a user's hand in the extending direction of a balloon catheter 1 or the longitudinal axis direction of a shaft 3, and a distal side refers to the opposite side of the proximal side, that is, the direction towards the person to be treated. The longitudinal axis direction of the balloon catheter 1 is preferably the same as the longitudinal axis direction x of a balloon body 20. In the present specification, even members other than long-shaped members are described as having the same longitudinal axis direction x. The radial direction y of the balloon body 20 is the direction perpendicular to the longitudinal axis direction x, connecting the center of the balloon body 20 and a point on the outer edge of the balloon body 20 in a cross-section perpendicular to the longitudinal axis direction x. The circumference direction z of the balloon body 20 is the direction along the outer edge of the balloon body 20 in a cross-section in the radial direction y.

As shown in FIG. 1, the balloon catheter 1 has the shaft 3 and a balloon 2 disposed at a distal end part of the shaft 3. The balloon catheter 1 can be configured such that fluid is introduced in the balloon 2 through the shaft 3, and the inflation and deflation of the balloon 2 can be controlled using an indeflator (pressurizer for balloons). The fluid may be a pressurized fluid pressurized by a pump or the lime.

The shaft 3 preferably has a flow path for the fluid inside, and further has a guidewire insertion path. Configurations in which the shaft 3 has an internal fluid path and guidewire insertion path inside include, for example, as shown in FIG. 1, a configuration where the balloon catheter 1 is of over-the-wire type, in which the balloon catheter 1 has a guidewire insertion path from the distal side to the proximal side of the shaft 3, the shaft 3 has an outer tube 31 and an inner tube 32, the inner tube 32 functions as a guidewire insertion path, and the space between the inner tube 32 and the outer tube 31 functions as a fluid flow path. In this configuration where the shaft 3 has the outer tube 31 and the inner tube 32, preferably, the inner tube 32 extends from the distal end of the outer tube 31 and penetrates distal to the balloon 2, the distal side of the balloon 2 is joined to the inner tube 32 and the proximal side of the balloon 2 is joined to the outer tube 31.

Alternatively, although not shown in the figures, the balloon catheter 1 in accordance with embodiments of the present invention may be of rapid-exchange type, in which a guidewire port is disposed at a midway from the distal side to proximal side of the shaft, and a guidewire insertion path is disposed from the guidewire port to the distal side of the shaft. In this case, the balloon catheter preferably has an outer shaft and an inner shaft that functions as the guidewire insertion path, and the space inside the outer shaft and outside the inner shaft preferably functions as the fluid flow path. Preferably, the inner shaft extends from the distal end of the outer shaft and penetrates the balloon, and the distal side of the balloon is joined to the inner shaft and the proximal side of the balloon is joined to the outer shaft.

As shown in FIG. 1 to FIG. 5, the balloon 2 for a balloon catheter has a straight tubular part 23, a proximal tapered part 22 located proximal to the straight tubular part 23, a proximal sleeve part 21 located proximal to the proximal tapered part 22, a distal tapered part 24 located distal to the straight tubular part 23, and a distal sleeve part 25 located distal to the distal tapered part 24; and has a balloon body 20 having an outer surface and an inner surface, and having a belt-like area 40 extending on the outer surface of the balloon body 20 in the longitudinal axis direction x of the balloon body 20; wherein the belt-like area 40 includes, in a cross-section in the radial direction y of the balloon body 20, at least one first region 41 consisting of a protrusion part 60 having a height H from the outer surface of the balloon body 20, and at least one second region 42 having a height from the outer surface of the balloon body 20 lower than the height H in a cross-section in the radial direction y; the first region 41 is disposed in the straight tubular part 23; and a surface roughness of the second region 42 is larger than a surface roughness of the first region 41. With this configuration, when the balloon 2 of the balloon catheter 1 is delivered to a stenosis, the second region 42 with a rougher surface can easily secure the balloon 2 to the stenosis site. Furthermore, since the height H of the first region 41 consisting of the protrusion part 60 is higher than the height h of the second region 42, the first region 41 can efficiently incise the stenosis. In addition, since the surface roughness of the first region is smaller than that of the second region, resistance when the protrusion part of the first region bites into the stenosis is suppressed and the stenosis can be easily incised by the protrusion part. As a result, according to the balloon 2, the stenosis can be efficiently incised while preventing the balloon 2 from shifting from the stenosis site, enabling easy and safe treatment.

As shown in FIG. 1 to FIG. 3, the balloon 2 has the straight tubular part 23, the proximal tapered part 22 located proximal to the straight tubular part 23, the proximal sleeve part 21 located proximal to the proximal tapered part 22, the distal tapered part 24 located distal to the straight tubular part 23, and the distal sleeve part 25 located distal to the distal tapered part 24. At least a part of the proximal sleeve part 21 and the distal sleeve part 25 can be fixed to the shaft 3. In the case where the balloon catheter 1 is of over-the-wire type and the shaft 3 has the outer tube 31 and the inner tube 32, at least a part of the proximal sleeve part 21 can be fixed to the outer tube 31 and at least a part of the distal sleeve part 25 can be fixed to the inner tube 32. Alternatively, in the case where the balloon catheter 1 of rapid-exchange type and the shaft 3 has the outer shaft and the inner shaft, at least a part of the proximal sleeve part 21 can be fixed to the outer shaft and at least a part of the distal sleeve part 25 can be fixed to the inner shaft.

Preferably, the proximal tapered part 22, the straight tubular part 23, and the distal tapered part 24 are the portions that are inflated when fluid is supplied to the interior of the balloon 2 through the shaft 3, while the proximal sleeve part 21 and the distal sleeve part 25 are not inflated when fluid is supplied to the interior of the balloon 2. This allows for stable fixation of the balloon 2 and the shaft 3 even when the balloon 2 is inflated.

The straight tubular part 23 preferably has the same diameter along the longitudinal axis direction x and has a cylindrical shape, while the proximal tapered part 22 and the distal tapered part 24 are preferably formed to reduce in diameter as they are away from the straight tubular part 23 and have a conical or conical trapezoidal shape. The reduced diameter of the proximal tapered part 22 and the distal tapered part 24 reduces the outer diameter of the proximal and distal end parts of the balloon 2 when the balloon 2 is deflated to reduce the step between the shaft 3 and the balloon 2, thereby making the balloon 2 easier to insert within the body cavity.

As shown in FIG. 2 to FIG. 5, the balloon body 20 has the outer surface and the inner surface, and has the belt-like area 40 extending on the outer surface of the balloon body 20 in the longitudinal axis direction x of the balloon body 20. The belt-like area 40 has a predetermined width in the circumferential direction z of the balloon body 20, and the width of the belt-like area is preferably 1/100 the circumference of the balloon body 20 or more, more preferably 1/80 or more, even more preferably 1/70 or more, and preferably ¼ or less, more preferably ⅛ or less, even more preferably 1/10 or less. The balloon body 20 may have a plurality of belt-like areas 40 in the circumferential direction z as shown in FIG. 1 to FIG. 5, or alternatively, although not shown in the figures, may have only one belt-like area 40. The number of the belt-like areas 40 in the circumferential direction z in the case of a plurality of belt-like areas 40 are disposed in the circumferential direction z is not particularly limited, and for example, preferably 2 or more, more preferably 3 or more, and may be 4 or more, and preferably 10 or less, more preferably 8 or less, and may be 6 or less. In this case, each of the plurality of belt-like areas 40 is preferably spaced apart in the circumferential direction z, and more preferably equally spaced apart in the circumferential direction z. Each of the plurality of belt-like areas 40 equally spaced apart facilitate securing of the balloon 2 and incision of the stenosis. The above range of the width of the belt-like area 40 shall apply to the sum of the widths of all the belt-like areas 40 when the plurality of belt-like areas 40 are disposed.

The belt-like area 40 is preferably provided continuously from the proximal end to the distal end of the balloon body 20. Within the belt-like area 40, the first region 41 and the second region 42 may be provided continuously or may be provided discontinuously.

The belt-like area 40 may be arranged parallel to the longitudinal axis direction x or may be arranged in a spiral shape around the outer surface of the balloon body 20 in the circumferential direction z. When the belt-like area 40 is arranged parallel to the longitudinal axis direction x, the second region 42 of the belt-like area 40 can secure the balloon 2 to the stenosis while the first region 41 allows the stenosis to be cut straight through. When the belt-like area 40 is arranged helically, the second region 42 of the belt-like area 40 can secure the balloon 2 to the stenosis while the first region 41 can incise the stenosis at an angle.

As shown in FIG. 4 and FIG. 5, the belt-like area 40 includes, in a cross-section in the radial direction y of the balloon body 20, at least one first region 41 consisting of the protrusion part 60 having the height H from the outer surface of the balloon body 20, and at least one second region 42 having the height h from the outer surface of the balloon body 20 lower than the height H in a cross-section in the radial direction y. The height H of the protrusion part 60 of the first region 41 is higher than the height h of the second region 42, allowing the stenosis to be easily incised by the first region 41 when the balloon 2 is delivered to the stenosis. In addition, the height H of the first region 41 can prevent the balloon 2 from stretching in the longitudinal direction x, and thus preventing the first region 41 from damaging blood vessels other than the stenosis. Furthermore, the first region 41 can improve pushability of the balloon 2.

The shape of the protrusion part 60 in a cross-section perpendicular to the longitudinal axis direction x may be any shape, and may be an approximate triangle as shown in FIG. 4, and may be, for example, a triangle, a square, a polygon, a semi-circle, part of a circle, an approximate circle, a fan shape, a wedge shape, a convex shape, a spindle shape, and combinations thereof. Triangles, squares, and polygons shall include those with clear corner vertices and straight edges, as well as so-called rounded polygons with rounded corners and those with curved edges at least partly. Alternatively, the cross-sectional shape of the protrusion part 60 may be irregularly shaped with bumps, chips, or the like. The maximum value of the height H of the protrusion part 60 is preferably 1 time the thickness of the balloon body 20 or higher, more preferably 1.5 times or higher, even more preferably 2 times or higher, and may be 50 times or lower, 30 times or lower, and 10 times or lower. The height H of the protrusion part 60 in the above range facilitates the incision of the stenosis by the first region 41, easily prevents stretching of the balloon 2 in the longitudinal axis direction x, and improve the pushability of the balloon 2.

The method of measuring the height H of the protrusion part 60 is described with reference to FIG. 4. UV-curable resin is introduced into the balloon 2 at 5 atmospheres to inflate the balloon 2, then UV is irradiated to cure the UV-curable resin, and the balloon body 20 is cut in the radial direction y. The cut surface is observed using a microscope such as an optical microscope to determine the radius rₒ of the outer circle Cₒ whose radius is the outer radius of the balloon body 20 in the radial direction y and the radius r_{cc} of the circumscribed circle CC of the protrusion part 60 sharing the center P with the outer circle Cₒ, and the height H of the protrusion part 60 is determined by subtracting the radius rₒ of the outer circle Cₒ from the radius r_{cc} of the circumscribed circle CC. The UV-curable resin can be any resin as long as it can be introduced to inflate the balloon 2.

The height h of the second region 42 can be determined in the same way as the height H of the protrusion part 60, as shown in FIG. 5. The height h of the second region 42 should be lower than the height H of the first region 41 consisting of the protrusion part 60, and the height h of the second region 42 is preferably ¾ the height H of the first region or lower, more preferably ½ or lower, and even more preferably ¼ or lower. The lower limit of the height h of the second region 42 may be 0, and may be a negative value, i.e., a concave shape of the outer surface of the portion of the balloon body 20 where the belt-like area 40 is arranged.

The first region 41 consisting of the protrusion part 60 is disposed in the straight tubular part 23. The first region 41 consisting of the protrusion part 60 may be disposed in the proximal tapered part 22 and/or the distal tapered part 24. The arrangement of the first region 41 in the straight tubular part 23 allows the straight tubular part 23, which has the largest diameter in the inflated state of the balloon 2, to make sufficient contact with the stenosis to easily incise the stenosis with the first region 41 disposed in the straight tubular part 23.

The surface roughness of the second region 42 is larger than the surface roughness of the first region 41. The larger surface roughness of the second region 42 allows the balloon 2 to be easily secured to the stenosis by the second region 42 when the balloon 2 is delivered to the stenosis. The smaller surface roughness of the first region 41 consisting of the protrusion part 60 makes it easier to cut into the calcified stenosis and plaque in the stenosis site and form a fissure, thereby preventing dissection of the vascular intima while dilating the stenosis. The surface roughness of the second region 42 is preferably 1.1 times the surface roughness of the first region 41 or larger, more preferably 1.25 times or larger, even more preferably 1.5 times or larger, and may be 100 times or smaller, 20 times or smaller, and 10 times or smaller.

The surface roughness is an arithmetic mean roughness Ra in a reference length of a roughness curve for the surfaces of the first region 41 and the second region 42. The arithmetic mean roughness Ra corresponds to an arithmetic mean roughness Ra specified in JIS B0601 (2001), and is measured according to JIS B0633 (2001). The reference length is as given in JIS B0633 (2001). For measurements, measuring instruments as specified in JIS B0651 (2001) (for example, a laser microscope manufactured by Keyence, VK-9510) are used.

In the case where the balloon body 20 has only one belt-like area 40, the surface roughness of the first region 41 and the second region 42 of the belt-like area 40 can be measured by the above method. In the case where the balloon body 20 has a plurality of belt-like areas 40 in the circumferential direction z, the surface roughness of the first region 41 and the second region 42 can be measured in the above manner for any one of the plurality of the belt-like areas.

Methods of making the height h of the second region 42 lower than the height H of the first region 41 include, for example, removing the protrusion part in the second region 42 using a laser, polishing the second region 42 using a grinder or the like, and crushing the protrusion part in the second region 42. Of these, the method of removing the protrusion part in the second region 42 using a laser is preferred, and in this case the use of a femtosecond laser with a short wavelength is more preferred. Whereas a laser with a longer wavelength would cause the temperature of the laser processing surface to rise and the resin in the second region 42 to melt, a femtosecond laser can remove the protrusion part of the second region 42 while suppressing the melting of the resin in the second region 42. Thus, a femtosecond laser can be used to form a structure in the second region 42 of fine irregularities with a period in the longitudinal axis direction x, i.e., repeating fine ridges and grooves parallel to the circumferential direction z, and the surface roughness of the second region 42 can be increased. Such a structure is suitable because the second region 42 makes it easier to secure the balloon 2 to the stenosis.

As shown in FIG. 5, the surface of the second region 42 is flat from a macroscopic point of view, and the fine irregularities are preferably formed on the flat surface. This allows the area of the second region 42 that is in contact with the lumen wall of the stenosis to become larger when the balloon 2 is delivered to the stenosis, which facilitates easier securing of the balloon 2 by the second region 42.

As shown in the straight tubular part 23 of FIG. 3 and FIG. 9, in the belt-like area 40, the first region 41 and the second region 42 may be formed so as to be discontinuously different in height. This is preferred because the balloon 2 can be secured to the stenosis also by the area where the height becomes discontinuously higher from the second region 42 to the first region 41.

Alternatively, as shown in FIG. 6 and FIG. 7, the belt-like area 40 may include at least one transition region 43 connecting the first region 41 and the second region 42. In the transition region 43, the height preferably increases continuously from the second region 42 to the first region 41. The surface roughness of the transition region 43 is preferably larger than the surface roughness of the first region 41. This allows the transition region 43 to secure the balloon 2 to the stenosis. In addition, the height continuously increasing from the second region 42 to the first region 41 by the transition region 43 can prevent the balloon 2 from snagging when inserted in the body cavity, facilitating insertion of the balloon 2. The surface roughness of the transition region 43 can be determined by the same measurement method as for the first region 41 and the second region 42.

The surface of the transition region 43 may be a flat slope, a concave curved surface, or a convex curved surface from a macroscopic point of view. In either case, the above effect can be achieved by the transition region 43.

The second region 42 is preferably disposed in at least one of the proximal sleeve part 21 and the distal sleeve part 25. The second region 42 disposed in both the proximal sleeve part 21 and the distal sleeve part 25, the second region 42 can secure the balloon to the stenosis site both when the balloon 2 is advanced and retracted. Alternatively, the second region 42 is disposed only in the proximal sleeve part 21 allows the balloon 2 to be secured to the stenosis site at the proximal end part of the balloon 2 while the distal end part to the inflatable part of the balloon 2 can incise the stenosis. In such a case, the distal end part, such as the distal sleeve part 25 and the distal tapered part 24, having the protrusion part 60 is effective for treatment in which the balloon catheter 1 creeps forward to incise and dilate the lesion. Alternatively, when the second region 42 is disposed only in the distal sleeve part 25, the balloon 2 can be secured to the stenosis site at the distal end part of the balloon 2, thus, allowing treatment without inadvertent advancement of the balloon 2. The second region 42 may be partially disposed in the longitudinal axis direction x, or may be disposed over the entire part in the longitudinal axis direction x in the proximal sleeve part 21 and/or the distal sleeve part 25, respectively.

As shown in FIG. 8, the belt-like area 40 preferably further includes a third region 45 having a height from the outer surface of the balloon body 20 higher than the height H of the protrusion part 60 of the first region 41 in a cross-section in the radial direction y of the balloon body 20, and when the second region 42 is disposed in at least one of the proximal sleeve part 21 and the distal sleeve part 25, the balloon 2 preferably satisfies at least one of the following (1) and (2):
(1) at least a part of the proximal tapered part 22 has the third region 45 when the second region 42 is disposed in the proximal sleeve part 21;
(2) at least a part of the distal tapered part 24 has the third region 45 when the second region 42 is disposed in the distal sleeve part 25.

Although FIG. 8 shows only the embodiment satisfying the above (2), the configuration satisfying the above (1) can be shown like the configuration satisfying the above (2). In addition, although FIG. 8 shows a configuration in which the distal sleeve part 25 has the third region 45 whose height gradually increases from the proximal side to the distal side than the height H of the protrusion part 60 of the first region 41, the height of the third region 45 need not increase gradually, as long as the distal sleeve part 25 has, at least partially, the third region 45 whose height is higher than the height H of the protrusion part 60 of the first region 41. This also the same for the proximal sleeve part 21 in the case (1).

The tapered parts that is connected to the sleeve part in which the second region 42 is formed has the third region 45 whose height is higher than the height H of the protrusion part 60, allowing the balloon 2 to be secured to the stenosis site by the second region 42 in the sleeve part, and also allowing the balloon 2 to be secured to the stenosis site by the third region 45 in the tapered part, and thus, stabilizing the securing of the balloon 2 in a synergistic effect. In this case, as shown in FIG. 8, the belt-like area 40 may have a transition region 43 connecting the first region 41 and the third region 45. The transition region 43 allows for easier securing of the balloon 2, and also improves the insertion of the balloon 2. Thus, the transition region 43 may be located not only at the portion connecting the first region 41 and the second region 42, but also at the portion connecting the third region 45 and the first region 41.

The surface roughness of the third region 45 may be larger than the surface roughness of the first region 41. This facilitates securing of the balloon 2 by the third region 45.

Alternatively, the surface roughness of the third region 45 may be smaller than the surface roughness of the second region 42. In this case, the surface roughness of the third region 45 may be the same as the surface roughness of the first region 41. Even if the surface roughness of the third region 45 is smaller than the surface roughness of the second region 42 like the first region 41, the third region 45 has a higher height than the height H of the protrusion part 60 of the first region 41, which can contribute to securing the balloon 2 to the stenosis site. In addition, the smaller surface roughness of the third region 45 can reduce the resistance of the third region 45 to bite into the stenosis, and the stenosis can be cut also by the third region 45.

The balloon 2 preferably satisfies at least one of the following (3) and (4):
(3) the second region 42 is disposed from the proximal sleeve part 21 to the proximal tapered part 22; and
(4) the second region 42 is disposed from the distal sleeve part 25 to the distal tapered part 24.

When the second region 42 is disposed in both sections from the proximal sleeve part 21 to the proximal tapered part 22 and from the distal sleeve part 25 to the distal tapered part 24, the second region 42 can secure the balloon 2 to the legion site both when the balloon 2 is advanced and retracted. Alternatively, when the second region 42 is disposed only from the proximal sleeve part 21 to the proximal tapered part 22, the proximal end part of the balloon 2 can be used to secure the balloon 2 to the stenosis site while the distal end part to the inflatable part of the balloon 2 can be used to cut the stenosis. In such a case, the distal end parts, such as the distal sleeve part 25 and the distal tapered part 24, having the protrusion part 60 is effective for treatment in which the balloon catheter 1 creeps forward to incise and dilate the lesion. Alternatively, when the second region 42 is disposed only from the distal sleeve part 25 to the distal tapered part 24, the distal end part of the balloon 2 can secure the balloon 2 to the stenosis site, allowing treatment without inadvertent advancement of the balloon 2.

The second region 42 may be partially disposed in the longitudinal axis direction x, or may be disposed over the entire part in the longitudinal axis direction x in the proximal tapered part 22 and/or the distal tapered part 24, respectively.

The second region 42 is preferably disposed in at least one of the proximal tapered part 22 and the distal tapered part 24. In this case, the second region 42 may not be disposed in the proximal sleeve part 21 and the distal sleeve part 25. This allows the surface roughness of the proximal sleeve part 21 and/or the distal sleeve part 25 to be reduced, and while the second region 42 disposed in the tapered part secures the balloon 2 to the stenosis site, the surface roughness of the portion that is the tip when the balloon 2 is advanced or retracted can be reduced, facilitating insertion of the balloon 2 in the body cavity.

As shown in FIG. 3, and FIG. 6 to FIG. 8, in the case where the second region 42 is formed in the tapered part and the sleeve part, it is preferred that one continuous second region 42 is formed. Alternatively, although not shown in the figures, two or more second regions 42 and the transition regions 43 may be formed discontinuously in the tapered part and the sleeve part.

The balloon 2 preferably satisfies at least one of the following (5) and (6):
(5) the second region 42 is disposed from the proximal tapered part 22 to a proximal end part of the straight tubular part 23; and
(6) the second region 42 is disposed from the distal tapered part 24 to a distal end part of the straight tubular part 23.

In this case, the second region 42 may not by disposed in the proximal sleeve part 21 and the distal sleeve part 25. This allows the surface roughness of the proximal sleeve part 21 and the distal sleeve part 25 to be reduced, while the balloon 2 can be secured to the stenosis site by the second region 42 disposed from the tapered part to the straight tubular part 23, the surface roughness of the portion that is the tip when the balloon 2 is advanced or retracted can be reduced, facilitating insertion of the balloon 2 in the body cavity. In addition, the placement of the second region 42 at the proximal and/or distal end part of the straight tubular part 23 allows the balloon 2 to be secured to the stenosis site by the straight tubular part 23, which has the largest diameter and can fully contact the stenosis, making the securing of the balloon 2 more stable.

The balloon 2 preferably satisfies at least one of the following (7) and (8):
(7) the second region 42 is disposed from the proximal sleeve part 21 to the proximal tapered part 22, and further to a proximal end part of the straight tubular part 23; and
(8) the second region 42 is disposed from the distal sleeve part 25 to the distal tapered part 24, and further to a distal end part of the straight tubular part 23.

This can further improve the effectiveness of the second region 42 in securing the balloon 2 to the stenosis site. When only the above (7) is satisfied, the distal end part, such as the distal sleeve part 25 and the distal tapered part 24, having the protrusion part 60 is effective for treatment in which the balloon catheter 1 creeps forward to incise and dilate the lesion. On the other hand, when only the above (8) is satisfied, the distal end part of the balloon 2 can be used to secure the balloon 2 to the stenosis site, making it easier to retract the balloon 2 after treatment without inadvertent advancement of the balloon 2.

As shown in FIG. 9, one or more of the second regions 42 are preferably disposed in the straight tubular part 23. This allows the balloon 2 to be secured to the stenosis site by the second regions 42 disposed in the straight tubular part 23, which has the largest diameter and can sufficiently contact the stenosis. In addition, because a portion where the height increases discontinuously from the second region 42 to the first region 41 is formed, the balloon 2 can be secured to the stenosis site by this portion as well, which is desirable. The number of the second region 42 formed in the straight tubular part 23 is 1 or more, more preferably 2 or more, even more preferably 3 or more, and may be 10 or less, 8 or less, and 6 or less. The total length in the longitudinal axis direction x of the second region 42 formed in the straight tubular part 23 is preferably 1/20 the length of the straight tubular part 23 in the longitudinal axis direction x or longer, more preferably 1/15 or longer, even more preferably 1/10 or longer, and preferably ¾ or shorter, more preferably ½ or shorter, and even more preferably ¼ or shorter.

As shown in FIG. 10, one or more of the transition regions 43 are preferably disposed in the straight tubular part 23. This allows the balloon 2 to be secured to the stenosis site by the second region 42 and the transition region 43 disposed in the straight tubular part 23, which has the largest diameter and can sufficiently contact the stenosis. In addition, because the height from the second region 42 to the first region 41 continuously increases owing to the transition region 43, it can be prevented for the balloon 2 from snagging when inserted into the body cavity, facilitating insertion of the balloon 2.

As shown in FIG. 9 and FIG. 10, in the embodiments where one or more of the second regions 42 and the transition regions 43 are disposed in the straight tubular part 23, the second region 42 and the transition region 43 are preferably disposed also in the tapered part and/or sleeve part. In this case, the second region 42 and the transition region 43 disposed in the tapered part and/or sleeve part may be arranged so that one of the second region 42 and the transition region 43 is continuous as shown in FIG. 9 and FIG. 10. Alternatively, although not shown in the figures, one or more of the second regions 42 or one or more of the second regions 42 and one or more of the transition regions 43 may be arranged also in the tapered part and/or sleeve part as in the straight tubular part 23. Alternatively, one or more of the second regions 42 or one or more of the second regions 42 and one or more of the transition regions 43 may be arranged in at lease one of the proximal tapered part 22 and the distal tapered part 24, and one second region 42 and one transition region 43 may be arranged in the sleeve part.

In the above embodiments, as shown in FIG. 10, it may be configured such that the plurality of transition regions 43 formed in the straight tubular part 23 are adjacent to each other and the protrusion part 60 of the straight tubular part 23 has an approximately V-shaped notches. In this case, the second region 42 can be interpreted as the boundary portion of the plurality of transition regions 43, i.e., the bottom portion of the approximately V-shape. Such a configuration allows easier incision of the stenosis by the protrusion part 60 of the first region 41, while the second region 42 and the transition region 43 secure the balloon 2 to the stenosis site.

As shown in FIG. 11, the belt-like area 40 in the second region 42 preferably has an inner protrusion part 61 that projects inwardly in the radial direction y from the inner surface of the balloon body 20 and extends in the longitudinal axis direction x of the balloon body 20. This allows the stiffness of the second region 42, which has the height h lower than the height H of the first region 41, to be increased, and it can be expected to prevent stretching of the balloon 2 in the longitudinal axis direction x and to improve the pushability of the balloon 2. The inner protrusion part 61 may or may not be formed in the first region 41.

Materials forming the balloon body 20 include, for example, polyolefin-based resin such as polyethylene, polypropylene, ethylene-propylene copolymer; polyester-based resin such as polyethylene terephthalate and polyester elastomer; polyurethane-based resin such as polyurethane and polyurethane elastomer; polyphenylene sulfide-based resin; polyamide-based resin such as polyamide and polyamide elastomer; fluorine-based resin; silicone-based resin; and natural rubber such as latex rubber. Only one of these may be used, or two or more may be used in combination. Of these, polyamide-based resin, polyester-based resin, and polyurethane-based resin are preferably used. In particular, elastomer resin is preferably used from the viewpoint of thinning and flexibility of the balloon body 20. For example, among polyamide-based resins, nylon 12, nylon 11, and the like are suitable for the resin forming the balloon body 20, and more preferably nylon 12 because it is relatively easy to mold when blow molding. Polyamide elastomers such as polyether ester amide elastomer and polyamide ether elastomer are also preferred in terms of thinning and flexibility of the balloon body 20. Of these, polyether ester amide elastomer is preferred in terms of high yield strength and good dimensional stability of the balloon body 20.

The protrusion part 60 of the first region 41, the second region 42, and the inner protrusion part 61 are preferably made of the same material as the balloon body 20. The protrusion part 60 of the first region 41, the second region 42, and the inner protrusion part 61 made of the same material as the balloon body 20 allows the protrusion part 60, the second region 42, and the inner protrusion part 61 to be less likely to damage the outer surface of the balloon body 20 while maintaining the flexibility of the balloon 2. The protrusion part 60, the second region 42, and the inner protrusion part 61 are preferably integrally molded with the balloon body 20. This can prevent the protrusion part 60, the second region 42, and the inner protrusion part 61 from falling off from the balloon body 20. Alternatively, the materials forming the protrusion part 60, the second region 42, and the inner protrusion part 61 may be different from the material forming the balloon body 20, as long as they are compatible to some degree with the material forming the balloon body 20.

The balloon 2 can be produced by placing a tubular parison 200 made of a resin, shown in FIG. 12, for example, in a mold having a groove on its lumen, followed by biaxially stretch-blow molding. The protrusion part 60 can be formed, for example, by inserting the parison 200 into the lumen of the mold such that a thick-walled portion 220 of the parison 200 is inserted into the groove of the mold, and then expanding the parison 200 by introducing fluid into a lumen 210 of the parison 200. The protrusion part 60 can then be left intact in the first region 41, and the second region 42 can be formed by lowering the height h of the second region 42 below the height H of the first region 41 by the method described previously. When forming the inner protrusion part 61, for example, the second region 42 and the inner protrusion part 61 can be formed by pressing the thick-walled portion 220 of the parison 200 against the groove-free portion of the mold and introducing a fluid into the lumen 210 of the parison 200 to expand the parison 200. The materials forming the parison 200 can be referred to the description of the materials forming the balloon body 20.

The shaft 3 is preferably made from resin, metal, or a combination of resin and metal. By using resin as the constituent material of the shaft 3, it is easier to provide flexibility and elasticity to the shaft 3. The use of metal as the constituent material of the shaft 3 improves the pushability of the balloon catheter 1. Resins forming the shaft 3 include, for example, polyamide-based resin, polyester-based resin, polyurethane-based resin, polyolefin-based resin, fluorine-based resin, polyvinyl chloride-based resin, silicone-based resin, and natural rubber. Only one of these may be used, or two or more may be used in combination. Of these, the material forming the shaft 3 is preferably at least one of polyamide-based resin, polyolefin-based resin, and fluorine-based resin. This can improve surface slipperiness of the shaft 3 and improve the insertion of the balloon catheter 1 into the body cavity. Metals forming the shaft 3 include, for example, stainless steel such as SUS304 and SUS 316, platinum, nickel, cobalt, chromium, titanium, tungsten, gold, Ni—Ti alloys, Co—Cr alloys, or combinations thereof.

The shaft 3 may be a single shaft 3 extending from the distal to the proximal side, or the shaft 3 may include a distal shaft and a proximal shaft, which are separate components, with the proximal end part of the distal shaft connected to the distal end part of the proximal shaft. The distal shaft and the proximal shaft may further comprise a plurality of tubular members. The configuration in which the shaft 3 consists of the distal shaft and the proximal shaft may include, for example, a configuration in which both the distal shaft and the proximal shaft are made of resin, and a configuration in which the distal shaft is made of resin and the proximal shaft is made of metal. In addition, the shaft 3 may have a laminated structure made of different or the same material.

The balloon 2 and the shaft 3 may be joined by adhesive bonding, welding, or by attaching a ring-shaped member at the point where the end of the balloon 2 and the shaft 3 overlap to swage them. Of these, the balloon 2 and the shaft 3 are preferably joined by welding. By welding the balloon 2 and the shaft 3, the bond between the balloon 2 and the shaft 3 is difficult to be released even when the balloon 2 is repeatedly inflated and deflated, easily increasing the strength of the bond between the balloon 2 and the shaft 3.

Although not shown in the figures, the balloon catheter 1 is preferably provided with a tip member at its distal end part. The tip member may be provided at the distal end part of the balloon catheter 1 by being connected to the distal end part of the balloon 2 as a separate component from the inner tube 32 or the inner shaft, or the inner tube 32 or the inner shaft extending distally beyond the distal end of the balloon 2 may function as the tip member.

Radiopaque markers may be placed on the inner tube 32 or the inner shaft inside the balloon 2 at the location of the balloon 2 in the longitudinal axis direction x, so that the position of the balloon 2 can be confirmed radiographically. The radiopaque markers are preferably placed at positions corresponding to both ends of the straight tubular part 23 of the balloon 2, or may be placed at a position corresponding to the center of the straight tubular part 23 in the longitudinal direction x.

As shown in FIG. 1, a hub 4 may be provided at a proximal side of the shaft 3, and the hub 4 may be provided with a fluid inlet 7 that is connected to the flow channel of the fluid supplied to the interior of the balloon 2. In addition, the hub 4 preferably has a guidewire insertion port 5 that is connected to the guidewire insertion channel. The balloon catheter 1 having the hub 4 provided with the fluid inlet 7 and the guidewire insertion port 5 can facilitate the operation of supplying fluid inside the balloon 2 to inflate and deflate the balloon 2 and delivering the balloon catheter 1 to a lesion site along a guidewire. The balloon 2 in accordance with embodiments of the present invention is applicable not only such a balloon catheter, which is of a so-called over-the-wire type, in which the guidewire is inserted over the distal to proximal side of the shaft, but also applicable to a so-called rapid-exchange balloon catheter, in which the guidewire is inserted from the distal side to the midway of the proximal side of the shaft. In the case of the rapid-exchange type, the hub 4 does not have to have a forked structure because the guidewire insertion port is provided halfway from the distal to the proximal side of the shaft.

The shaft 3 and the hub 4 may be joined by, for example, adhesive bonding or welding. Of these, the shaft 3 and the hub 4 are preferably joined by adhesive bonding. The adhesive bonding of the shaft 3 and the hub 4 can increase the bonding strength of the shaft 3 and the hub 4 to increase durability of the balloon catheter 1 when the materials forming the shaft 3 and the hub 4 are different, for example, in a case where the shaft 3 is made of material having high flexibility and the hub 4 is made of material having high stiffness.

In the case where the balloon catheter 1 is of over-the-wire type, the outer wall of the outer tube 31 is preferably coated as appropriate. In the case of a rapid-exchange type, the outer walls of the distal shaft and/or proximal shaft are preferably coated as appropriate, and more preferably both the distal shaft and the proximal shaft are coated.

The coating can be a hydrophilic or hydrophobic coating, depending on the purpose, and can be applied by dipping the shaft 3 into a hydrophilic or hydrophobic coating agent, applying a hydrophilic or hydrophobic coating agent to the outer wall of the shaft 3, or coating the outer wall of the shaft 3 with a hydrophilic or hydrophobic coating agent. The coating agent may contain medical agents and additives.

Hydrophilic coating agents include hydrophilic polymers such as polyvinyl alcohol, polyethylene glycol, polyacrylamide, polyvinyl pyrrolidone, methyl vinyl ether maleic anhydride copolymer, and hydrophilic coating agents made of any combination thereof.

Hydrophobic coating agents include polytetrafluoroethylene (PTFE), ethylene-propylene fluoride (FEP), perfluoroalkoxy alkane (PFA), silicone oil, hydrophobic urethane resin, carbon coating, diamond coating, diamond-like carbon (DLC) coating, ceramic coating, and substances with low surface free energy terminated with an alkyl group or a perfluoroalkyl group.

The present application claims priority based on Japanese Patent Application No. 2021-182088 filed on Nov. 8, 2021. All the contents described in Japanese Patent Application No. 2021-182088 filed on Nov. 8, 2021 are incorporated herein by reference.

### DESCRIPTION OF REFERENCE SIGNS

1: balloon catheter
2: balloon
3: shaft
4: hub
5: guidewire insertion port
7: fluid inlet
20: balloon body
21: proximal sleeve part
22: proximal tapered part
23: straight tubular part
24: distal tapered part
25: distal sleeve part
31: outer tube
32: inner tube
40: belt-like area
41: first region
42: second region
43: transition region
45: third region
60: protrusion part
61: inner protrusion part
200: parison
210: lumen of the parison
220: thick-walled portion of the parison
Cₒ: outer circle
CC: circumscribed circle of the protrusion part
rₒ: radius of the outer circle Cₒ
r_{cc}: radius of the circumscribed circle CC
H: height of the first region
h: height of the second region
x: longitudinal axis direction of the balloon body
y: radial direction of the balloon body
z: circumferential direction of the balloon body

1. A balloon for a balloon catheter, comprising a balloon body having a straight tubular part, a proximal tapered part located proximal to the straight tubular part, a proximal sleeve part located proximal to the proximal tapered part, a distal tapered part located distal to the straight tubular part, and a distal sleeve part located distal to the distal tapered part; wherein
the balloon body has an outer surface and an inner surface, and a belt-shaped area extending on the outer surface of the balloon body in a longitudinal axis direction of the balloon body;
the belt-shaped area includes, in a cross-section in a radial direction of the balloon body, at least one first region including a protrusion part having a height H from the outer surface of the balloon body, and at least one second region having a height from the outer surface of the balloon body lower than the height H;
the first region is disposed on the straight tubular part; and
a surface roughness of the second region is larger than a surface roughness of the first region.

2. The balloon for the balloon catheter according to claim 1, wherein the belt-shaped area includes at least one transition region connecting the first region and the second region, and a surface roughness of the transition region is larger than the surface roughness of the first region.

3. The balloon for the balloon catheter according to claim 1, wherein the second region is disposed on at least one of the proximal sleeve part and the distal sleeve part.

4. The balloon for the balloon catheter according to claim 3, wherein the belt-shaped area further includes a third region having a height from the outer surface of the balloon body higher than the height H of the protrusion part of the first region in a cross-section in the radial direction of the balloon body, and the balloon satisfies at least one of the following (1) and (2):
(1) at least a part of the proximal tapered part has the third region, and the second region is disposed on the proximal sleeve part; and
(2) at least a part of the distal tapered part has the third region, and the second region is disposed in the distal sleeve part.

5. The balloon for the balloon catheter according to claim 1, wherein the balloon satisfies at least one of the following (3) and (4):
(3) the second region is disposed to extend from the proximal sleeve part to the proximal tapered part; and
(4) the second region is disposed to extend from the distal sleeve part to the distal tapered part.

6. The balloon for the balloon catheter according to claim 1, wherein the second region is disposed on at least one of the proximal tapered part and the distal tapered part.

7. The balloon for the balloon catheter according to claim 1, wherein the balloon satisfies at least one of the following (5) and (6):
(5) the second region is disposed to extend from the proximal tapered part to a proximal end part of the straight tubular part; and
(6) the second region is disposed to extend from the distal tapered part to a distal end part of the straight tubular part.

8. The balloon for the balloon catheter according to claim 1, wherein the balloon satisfies at least one of the following (7) and (8):
(7) the second region is disposed to extend from the proximal sleeve part to the proximal tapered part, and further to a proximal end part of the straight tubular part; and
(8) the second region is disposed to extend from the distal sleeve part to the distal tapered part, and further to a distal end part of the straight tubular part.

9. The balloon for the balloon catheter according to claim 1, wherein one or more of the second regions are disposed on the straight tubular part.

10. The balloon for the balloon catheter according to claim 1, wherein the belt-shaped area includes at least one transition region connecting the first region and the second region, and one or more of the transition regions are disposed in the straight tubular part.

11. The balloon for the balloon catheter according to claim 1, wherein the belt-shaped area has an inner protrusion part that projects inwardly in a cross-section in the radial direction from the inner surface of the balloon body.
